(19) United States
(12) Patent Application Publication
(10) Pub. No.: US 2010/0272773 A1
(43) Pub. Date: Oct. 28, 2010

(54) Use of Drug Polymorphs to Achieve Controlled Drug Delivery From a Coated Medical Device

(75) Inventors: Steve Kangas, Woodbury, MN (US); Yen-Lane Chen, New Brighton, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/765,522

(22) Filed: Apr. 22, 2010

Related U.S. Application Data
(60) Provisional application No. 61/172,629, filed on Apr. 24, 2009.

(51) Int. Cl. A61K 31/337; A61K 9/00; A61P 35/00; A61M 25/10

(52) U.S. Cl. 424/422; 514/449; 604/103.02

(57) ABSTRACT

When making a medical device having a drug coating thereon, the drug having a plurality of characteristic morphological forms, the manufacturing process is controlled to produce a predetermined ratio of said morphological forms on the device. The process has application to drug coated balloons.

### BACKGROUND OF THE INVENTION

Balloons coated with paclitaxel containing formulations are known. In some cases paclitaxel has been applied directly to the balloon or to a coating placed on the balloon. In other cases paclitaxel has been formulated with an excipient that may be polymer, a contrast agent, a surface active agent, or other small molecules that facilitate adhesion to the balloon and/or release from the balloon upon expansion. The formulations have typically been applied from solution, and may be applied to the entire balloon or to a folded balloon, either by spraying, immersion or by pipette along the fold lines.

Paclitaxel coated balloons that provide high release rates from the balloon surface have recently been developed. However these balloons do not yet provide for delivery of predictable amounts of the drug to the tissue at the delivery site nor do they provide for a predictable therapeutic drug tissue level over an extended time period.

### SUMMARY OF THE INVENTION

Heretofore the form that the drug takes on the balloon has not been a subject of concern for drug coated balloons. The present invention recognizes that for consistent drug release profile, however, it is important to control the polymorph composition of the drug.

In one aspect the invention pertains to a method of making a medical device having a drug coating thereon wherein the drug has a plurality of characteristic morphological forms wherein the process is controlled to produce a predetermined ratio of said morphological forms on the device.

In another aspect the invention pertains to a method of controlling tissue residence of a drug delivered by a transient device that is inserted into a body passageway, advanced through the body passageway to a treatment site and delivers drug to tissue at the site and is removed, wherein the drug has at least two morphological forms having different tissue residence characteristics, wherein the ratio of said morphological forms is controlled to provide therapeutically effective dosage at the site of delivery for a predetermined time after delivery. In some embodiments the ratio is predetermined to provide a tissue residence of a therapeutically effective dosage for an extended period of time, for instance 5 days, 10 days, 20 days, 30 days or 40 days after delivery. In some embodiments the drug is provided as a mixture at least two different morphological forms. In some embodiments the ratio is predetermined to provide a tissue residence of a therapeutically effective dosage for an extended period of time, for instance 5 days, 10 days, 20 days, 30 days or 40 days after delivery.

In another aspect the invention pertains to a drug coated balloon comprising a layer comprising a drug that has a plurality of morphological forms, the balloon having a selected morphological form or a selected mixture of said morphological forms distributed uniformly over the surface of the balloon.

In another aspect the invention pertains to a drug coated balloon wherein the drug is paclitaxel or a mixture of paclitaxel and at least one other drug, the balloon having a selected distribution of at least two different morphological forms of paclitaxel thereon.

Still other aspects of the invention are described in the Figures, the Detailed Description of Preferred Embodiments and/or in the Claims below.

### BRIEF DESCRIPTION OF THE FIGURES

FIG. 1 Photograph: Drug coated balloon of prior art after deployment

FIG. 2 Graph: Particle size distribution of coating ejected from prior art balloon during deployment.

FIG. 3 Photograph: Clear polyurethane tube after deployment of state of prior art drug coated balloon showing drug particles on the ID of the tubing.

FIG. 4 Diagram showing polymorphs of PTx

FIG. 5. SEM of PTx coated from 40/60 THF/EtOH per embodiment 1.

FIG. 6 Ptx coated balloon. Ptx coated from DMSO per embodiment 2, Method 1.

FIG. 7 Ptx coated balloon. Ptx coated from DMSO, per embodiment 2, Method 1.

FIG. 8 SEM—EtOH vapor annealed balloons, per embodiment 2, Method 2.

FIGS. 9a-9c Show SEM of the coated balloon, the tube after deployment and the filter after soak and deploy, respectively, per embodiment 6.

FIG. 10a SEM of PTx coated from 1:1 THF:Toluene, per embodiment 7.

FIG. 10b Deploy in Tube image, per embodiment 7.

FIG. 10c Deploy in tube—high mag image, per embodiment 7.

FIG. 11 SEM images (1,000×) of PTx coated from different ratios of THF/EtOH, per embodiment 8.

FIG. 12a SEM Ptx coated from 20/80 THF/EtOH—vapor annealed in EtOH, per embodiment 9.

FIG. 12b SEM Ptx coated from 40/60 THF/EtOH—vapor annealed in EtOH, per embodiment 9.

FIG. 13a SEM of PTx/PVP coating (2000×), per embodiment 10.

FIG. 13b Deploy in tube images Ptx/PVP coating, per embodiment 10.

FIG. 13c Filtered particles image from soak and deploy Ptx/PVP, per embodiment 10.

FIG. 14a SEM image of Ptx/PVP from embodiment 10 after EtOH solvent annealing, per embodiment 11.

FIG. 14b Deploy in tube image of Ptx/PVP from embodiment 10 after EtOH solvent annealing, per embodiment 11.

FIG. 14c High mag deploy in tube image, per embodiment 11.

FIG. 14d Soak and deploy filter images of Ptx/PVP from example 5 after EtOH solvent annealing, per embodiment 11.

FIG. 15a SEM image of PTx/PVP (55K MW) coating coated from 40/60 THF/EtOH, per embodiment 12.

FIG. 15b SEM image of PTx/PVP coating (1.3M MW) coated from 40/60 THF/EtOH, per embodiment 12.

FIG. 15c Deploy in tube images of PTx/PVP (55K MW) coating coated from 40/60 THF/EtOH, per embodiment 12.

### DETAILED DESCRIPTION OF PREFERRED EMBODIMENTS

Drugs such as paclitaxel (“PTx”) have more than one morphological form. In the case of paclitaxel, amorphous, anhydrous crystalline, crystalline dihydrate and dehydrated forms are known. These have different solubilities and dissolution rates in aqueous fluids, including blood. For medical devices such as drug coated balloons in which the drug is delivered to tissue without regulation of an elution coating, the reproducibility of drug delivery to the depends in part on physical characteristics of the drug layer, but also on the ability to reliably produce specific polymorph form(s) or distribution provided on the device. Further the ability to provide drug delivery over extended time depends on the ability to provide a desired polymorph distribution.

In some embodiments the drug is a lipophilic substantially water insoluble drug, such as paclitaxel, rapamycin, everolimus, or another drug that inhibits restenosis. Other drugs that may be suitable are described in documents identified later herein. Mixtures of drugs, for instance paclitaxel and rapamycin, may be employed.

According to the invention the drug is one that has polymorph forms, i.e. at least two characterizable morphologies that have different solubilities, or crystal forms. The drugs which can be used in embodiments of the present invention, can be any therapeutic agent or substance that has therapeutic benefit for local administration by delivery from a medical device inserted into the body and that also exists in polymorph forms.

In at least some embodiments the different morphological forms have characteristics that affect tissue uptake of the drug at the delivery site.

In some embodiments the drugs are deliverable from the surface of catheter balloons. In some embodiments the drugs are deliverable on stents or other devices implanted or left in place for extended times in the body. In other embodiments the drugs are deliverable by perfusion catheters to a localized site.

In some embodiments the drug is applied to a device, such as a balloon, that provides transient contact delivery of the drug directly to tissue without use of a release regulating polymer, such as is typically present on drug eluting stents or in microencapsulated drug particles.

In some embodiments the drug may be coated with a protective polymeric layer that functions to reduce loss during deployment of the device to the site of administration, but that substantially disintegrates in the course of the deployment or during transfer of the drug from the device at the site of administration. Suitably such protective layer has a thickness of 0.5 μm or less, 0.1 μm or less, or 0.01 μm or less. Polymers or copolymers that have a good solubility in water and a molecular weight sufficient to slow dissolution of the coating enough to provide practical protection may be used. Other protective layers may be effective if they break up into fine particles during drug delivery, for instance upon balloon expansion. Protective coating thickness may be adjusted to give an acceptable dissolution and/or degradation profile.

In some embodiments the drug is formulated with an excipient. An excipient is an additive to a drug-containing layer that facilitates adhesion to the balloon and/or release from the balloon upon expansion. The excipient may be polymer, a contrast agent, a surface active agent, or other small molecule. In at least some embodiments the drug is substantially insoluble in the excipient.

In some embodiments the excipient may remain on the delivery device at the time of drug transfer but allow efficient transfer of the drug from the mixture. In some embodiments the excipient provides weak phase boundaries with the drug particles that are easily overcome when a balloon is expanded, regardless of whether the excipient remains on the device or initially leaves the device with the drug. In some embodiments the excipient substantially degrades or dissolves in the course of the deployment or during transfer of the drug from the device at the site of administration such that little or none of the excipient is detectable on the tissue after a short interval, for instance an interval of 2 days, 1 day, 12 hours, 4 hours, 1 hour, 30 minutes, 10 minutes or 1 minute. In some embodiments dissolution or degradation of the excipient during deployment provides porosities in the drug-containing layer by the time the device is at the site of administration.

Examples of excipients that may be employed include polymeric and non-polymeric additive compounds, including polyvinylpyrrolidone (PVP), sugars such as mannitol, contrast agents such as iopamide, citrate esters such as acetyltributyl citrate, and pharmaceutically acceptable salts.

In some embodiments the drug containing layer is applied over an underlayer of material that has a high solubility in bodily fluids to undercut the drug facilitate breakup of the drug-containing layer upon balloon expansion. An example of a suitable underlayer material is pectin.

Numerous other excipients and additive compounds, protective polymer layers, underlayer materials and drugs are described in one or more of the following documents:
U.S. Pat. No. 5,102,402, Dror et al (Medtronic, Inc.)
U.S. Pat. No. 5,370,614, Amundson et al, (Medtronic, Inc.)
U.S. Pat. No. 5,954,706, Sahatjian (Boston Scientific Corp)
WO 00/32267, SciMed Life Systems; St Elizabeth's Medical Center (Palasis et al)
WO 00/45744, SciMed Life Systems (Yang et al)
R. Charles, et al, “Ceramide-Coated Balloon Catheters Limit Neointimal Hyperplasia After Stretch Injury in Cartoid Arteries,” Circ. Res. 2000; 87; 282-288 U.S. Pat. No. 6,306,166, Barry et al, (SciMed Life Systems, Inc.)
US 2004/0073284, Bates et al (Cook, Inc; MED Inst, Inc.)
US 2006/0020243, Speck
WO 2008/003298 Hemoteq AG, (Hoffman et al)
WO 2008/086794 Hemoteq AG, (Hoffman et al)
US 2008/0118544, Wang
US 20080255509, Wang (Lutonix)
US 20080255510, Wang (Lutonix)
   All incorporated herein by reference in their entirety.

According to an embodiment the invention the drug is provided on the device in a manner that is controlled to produce a predetermined ratio of said morphological forms.

In some cases paclitaxel has been applied directly to the balloon or to a coating placed on the balloon. In other cases paclitaxel has been formulated with an excipient that may be polymer, a contrast agent, a surface active agent, or other small molecules that facilitate adhesion to the balloon and/or release from the balloon upon expansion. The formulations have typically been applied from solution, and may be applied to the entire balloon or to a folded balloon, either by spraying, immersion or by pipette along the fold lines.

Drugs such as paclitaxel have more than one morphological form. In the case of paclitaxel, amorphous, anhydrous crystalline, crystalline dehydrate, dehydrated forms and Pam forms are known. These have different solubilities and dissolution rates in aqueous fluids, including blood. For medical devices such as drug coated balloons in which the drug is delivered to tissue without regulation of an elution coating, the reproducibility of drug delivery to the depends in part on physical characteristics of the drug layer, but also on the ability to reliably produce specific polymorph form(s) or distribution provided on the device. Further the ability to provide drug delivery over extended time depends on the ability to provide a desired polymorph distribution.

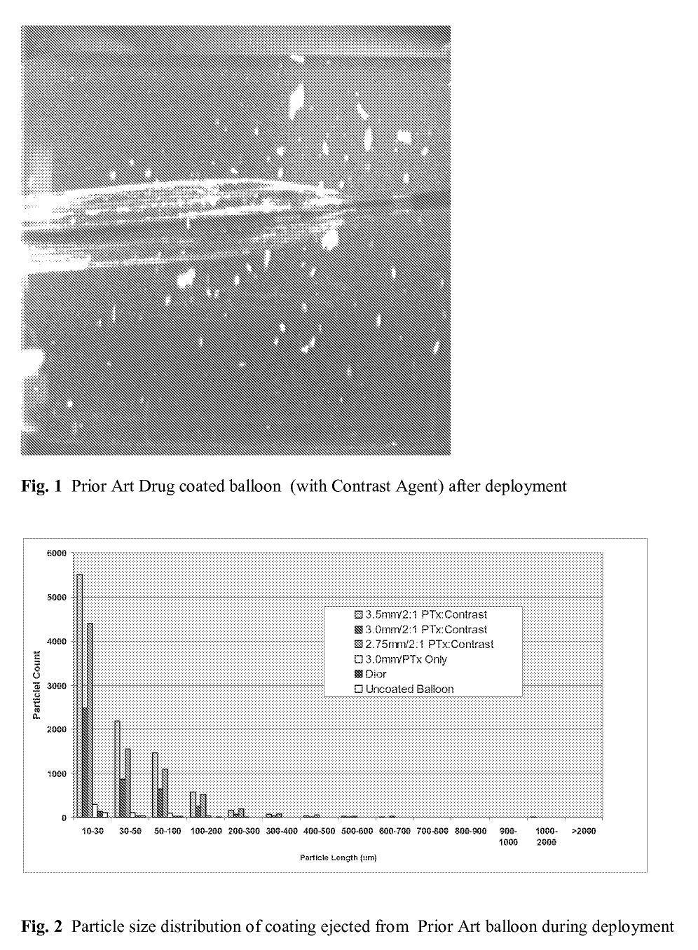

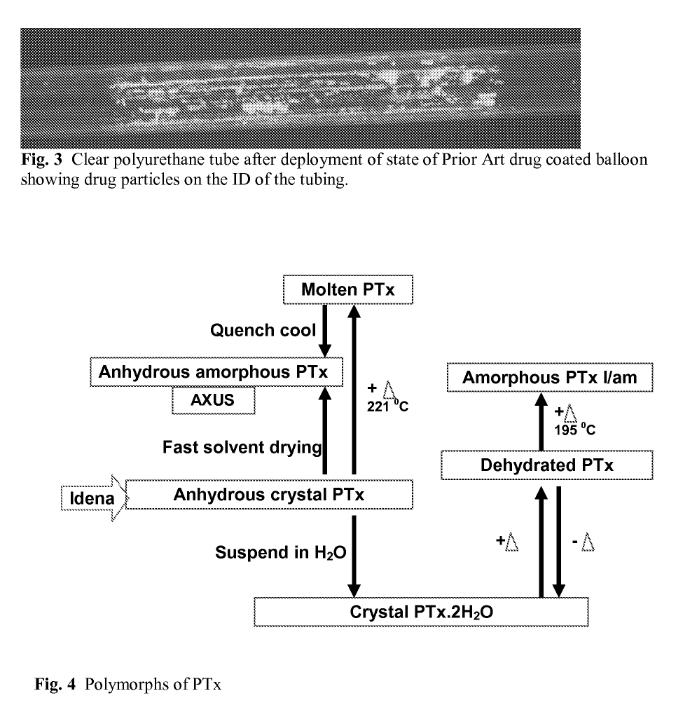

FIG. 1 is a photograph showing a drug coated balloon from one prior art source that was deployed in a clear polyurethane tubular system designed to mimic aspects of vascular deployment, after travel to a deployment site and inflation. Additional analysis of these balloons and their deployment lead the inventors to the following conclusions:
The balloon coating is comprised of a blend of PTx and contrast (Iopromide). The drug and contrast are for the most part immiscible and form a two phase blend. Coatings of both PTx and Iopromide are stiff solid film (high glass transition temperatures for both drug and contrast). Owing to their low molecular weight of both materials, the coatings are very brittle with poor cohesive strength.
The resulting ejected coating is in the form of particulates with a broad distribution of particle sizes (from <10 um to >500 um). See FIG. 2. These particulates are embedded into the artery during deployment (see FIG. 3 photo of polyurethane tube in which a DCB has been deployed).
Upon deployment of the folded balloon, the coating is placed under significant bending stress. As a result the coating cracks and is released from the balloon.
The resulting ejected coating is in the form of particulates with a broad distribution of particle sizes (from <10 um to >500 um). These particulates are embedded into the artery during deployment.

The inventors hereof have recognized that solid particulates on the artery wall have 3 potential fates—some are likely flushed from the artery wall into the blood stream.

Those that remain in contact with the artery wall will slowly dissolve—some fraction dissolving into the blood stream and some fraction taken up by the vessel (the therapeutic dose). Very small particles <1 um can be taken up directly into the arterial tissue. Some of the drug that diffuses into the vessel wall binds to and stabilizes the cell microtubules, thereby affecting the restenotic cascade after injury of the artery.

The size, distribution and extent of crystallinity of the drug particles of prior art balloons is poorly controlled. However these factors will play a critical role in tissue uptake and duration of arterial tissue levels. Methods to control these factors therefore are be important in designing drug eluting balloons.

Paclitaxel is known to have several polymorphs. These polymorphs and are shown in FIG. 5. The PTx polymorphs have different solubility and other physical chemical properties. Table 1 shows the solubility of 3 polymorphs of PTx.

TABLE 1
PTx Polymorph Solubility

| PTx | Solubility in H₂O | Dissolution rate |
|---|---|---|
| Solid State | (μg/ml) | (μg/ml/hr) |
| PTx Amphorous | 6 | — |
| PTx Anhydrous Crystal | 3 | 0.95 |
| PTx 2H2O | 0.75 | 0.10 |
| Crystalline | | |

The ability to control the Ptx morphology on a drug coated balloon is important in achieving proper dosing. This is illustrated by the following example. Based on published preclinical data, for a prior art balloon coated with 450 μg Ptx, typically one observes about 5% transfer efficiency of solid Ptx particles to the vessel (˜23 μg). If the Ptx transferred to the vessel is anhydrous crystalline then it will take about 1 day for complete dissolution of the Ptx (23 μg/0.95 μg/mL/hr). The Ptx duration is far too short to be efficacious. If the Ptx on the DEB is crystalline dehydrate then it will take about 10 days for complete dissolution (23 μg/0.1 μg/mL/hr)—a duration that will be more efficacious. Other factors such as particle size will also influence dissolution. The objective of this simple calculation is to highlight the potential impact of PTx polymorphs on DEB performance and the importance of understanding and being able to control the morphology.

In addition to creating DEB coatings of specific Ptx polymorphs it is desirable to prepare a balloon coating that possesses a blend of Ptx polymorphs. For example it will be advantageous to have both amorphous and crystalline morphologies within the same coating. The faster dissolving amorphous Ptx will provide for initial burst release to the vessel and crystalline phase(s) will provide for slower dissolution into the vessel for sustained tissue levels. This can be accomplished for example by 1^{st} generating an amorphous coating. Subjecting the coated balloon to solvent vapor (e.g. ethanol vapor) for time intervals less than required to achieve 100% crystallinity will lead to a coating with a mix of amorphous and crystalline phases. If the anhydrous crystalline phase is the initial crystalline phase produced, further treatment of the balloon at high humidity for specific times will convert a percentage of the anhydrous crystalline Ptx to the dihydrate. The ratio of conversion to the dihydrate is controlled by dwell time at high humidity and so the dehydrate can be controlled to a desired fraction as well. A specific rate of drug release from DEB coating may be tailored by varying the ratio of these three Ptx polymorphs with different solubility and dissolution rates on a single coating.

In some cases conversion of PTx on a balloon to the dehydrate is also practical and so the properties of that polymorph can also be utilized in the invention. Further the invention has application to other devices that may be used for direct delivery of the drug to a treatment site in the body. If the device can withstand the temperatures needed to produce them both the dehydrate and the semicrystalline amorphous PTx Pam can be utilized in addition to the amorphous, anhydrous crystalline and dehydrate crystalline forms.

The devices of the present invention, may be deployed in vascular passageways, including veins and arteries, for instance coronary arteries, renal arteries, peripheral arteries including illiac arteries, arteries of the neck and cerebral arteries, and may also be advantageously employed in other body structures, including but not limited to arteries, veins, biliary ducts, urethras, fallopian tubes, bronchial tubes, the trachea, the esophagus and the prostate.

In some embodiments a drug coating of paclitaxel on a balloon contains from 100 to 1000 μg of paclitaxel, for instance 200-800 μg, 300-600 μg, or 400-500 μg of paclitaxel. In some embodiments the amount of amorphous paclitaxel on the balloon is from 0-80 μg, less than 60 μg, or less than 30 μg, with the remaining being one or both crystalline forms. In some embodiments the amount of anhydrous crystalline paclitaxel on the balloon is from 0-200 μg, less than 100 μg, or less than 50 μg. In some embodiments the amount of crystalline dihydrate paclitaxel on the balloon is from 50 to 1000 μg, 100-800 μg, 200-600 μg, 300-500 or 350-450 μg. In some embodiments the fraction of amorphous paclitaxel in the coating is from 0-25%, for instance about 1%, about 2%, about 3%, about 5%, about 6%, about 8%, about 10%, about 12%, about 15%, about 18%, about 20%, about 22%, or about 25%, based on total paclitaxel weight. In some embodiments the fraction of anhydrous crystalline paclitaxel is from 0% to about 99%, for instance 1-95%, 5-80%, about 1%, about 2%, about 3%, about 5%, about 6%, about 8%, about 10%, about 15%, about 20%, about 25%, about 30%, about 40%, about 50%, about 60%, about 70%, or about 80%, based on total paclitaxel weight. In some embodiments the fraction of dihydrate crystalline paclitaxel is from 1% to 100%, for instance 1-99%, 5-95%, about 10%, about 15%, about 20%, about 25%, about 30%, about 35%, about 40%, about 45%, about 50%, about 55%, about 60%, about 65%, about 70%, about 75%, about 80%, about 85%, about 88%, about 89%, about 90%, about 91%, about 92%, about 93%, about 94%, about 95%, about 96%, about 97%, about 98%, or about 99%, based on total paclitaxel weight.

The present invention also describes methods of changing the coating morphology to control the break-up (particle size) and crystallinity of the coating. Control of coating morphology is accomplished by the choice of solvents used to coat the drug/excipient. This involves utilizing a fast evaporating good solvent for the drug and a second slower evaporating solvent that is a poor solvent for the drug. Typically most coatings, e.g. architectural and drug eluting stent coatings, are formulated using good solvents to achieve good coating quality (i.e. smooth, continuous). For example if one coats Ptx or Ptx/excipient from a solvent that is a fair-good solvent for PTx and the excipient, the resulting coating is continuous/smooth glassy coating. It has been shown that such balloon coatings break into quite large particles when deployed in a vessel (synthetic tube or ex-vivo artery). In the case of drug eluting balloons therefore there is a need to be able to control the coating morphology to achieve various discontinuous or porous coatings that lead to smaller more repeatable particles during deployment of the balloon.

It has been found that if one coats drug from a mixture of a fast evaporating good solvent and a slower evaporating poor solvent that during drying the drug precipitates, resulting in a porous coating. With certain solvents one can even generate monodisperse spherical drug particles during coating/drying process. By varying the solvents and solvent ratioone can obtain a range of coating porosities and hence particle sizes. Also, when drug crystallinity is induced, e.g. by vapor treatment of the drug coating, the crystal sizes can be altered by the selection of initial coating morphology and solvent selection. Thus for a specific coating formulation one can generate coatings that range from amorphous to crystalline, with different particle sizes.

The following non-limiting embodiments illustrate methods to achieve various PTx polymorphs on Drug Eluting Balloons and to control the coating morphology:

### 1. Amorphous Microporous Ptx

A folded coronary angioplasty balloon (Liberte) is inflated at low pressure to achieve it's inflated profile. A solution of Paclitaxel (10-20 wt % solids) in 40/60 (wt/wt) THF/EtOH is prepared. The balloon catheter is dipped into the PTx solution and withdrawn at a rate of 0.3-1 in/sec. The balloon is allowed to dry at room temperature. The coating dries very rapidly at room temperature (seconds), thus resulting in “quenching” PTx in the amorphous state. FIG. 5 shows SEM image of the coated balloon. Coating from THF/EtOH results in a microporous amorphous coating.

Alternately the PTx can be applied to the balloon via spray coating process.

### 2. Anhydrous Crystalline Ptx

### Method 1. Crystallization Through Controlled Drying.

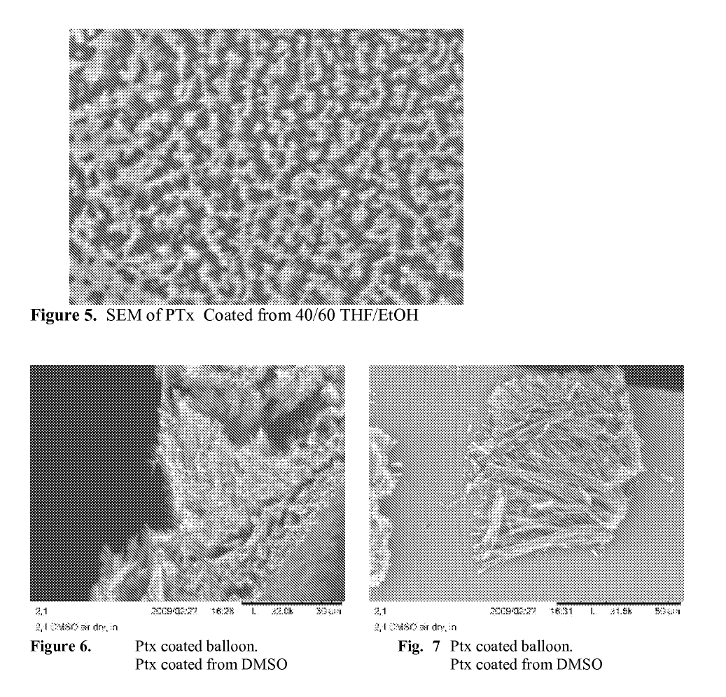

PTx is dissolved in anhydrous DMSO to make a solution of 5-20% Ptx (wt). DMSO is a slow evaporating solvent at room temperature and thus allows slow crystallization of PTx. A folded balloon catheter is dip coated in the PTx/DMSO solution and allowed to dry at room temperature for 24 hours. FIGS. 6 and 7 show SEM of the cross-sectioned balloon showing the presence of fine hair-like PTx crystals. Alternatively one can manipulate the coating process to control the drying rate—for example one could use faster drying solvents such as EtOH but dry at low temperature (0-50° F.) for slower solvent evaporation which allows time for crystallization of PTx.

### Method 2. Solvent Vapor Annealing

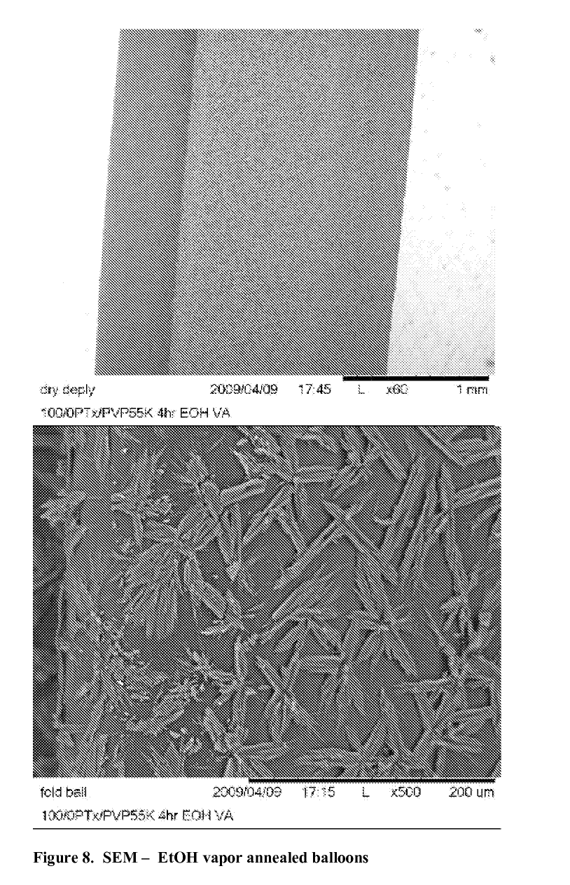

The coated balloon from embodiment 1 is placed in a sealed container at room temperature containing saturated ethanol vapor for 4 hrs. The amorphous PTx converts to crystalline form in the ethanol vapor environment. Representative SEM images of the vapor annealed balloon coating are shown in FIG. 8.

### 3. Crystalline Dihydrate

The Ptx dihydrate can be prepared by the following methods:

### Method 1. Treatment in Water

The coated balloon of embodiment 2 is placed in water at room temperature for 24 hrs. This will convert the anhydrous Ptx to the dihydrate.

### Method 2. Treatment at High Humidity

The coated balloon of embodiment 2 is placed in a humidity chamber at 25-50° C. and 90-95% RH for 24 hours.

Method 3. Coating Ptx from Organic Solvent+Water

The balloon can be coated as described in embodiment 2, method 1 but with the addition of water to the coating solvent, for instance 1-33%, about 1%, about 3%, about 5%, about 8%, about 10%, about 12%, about 15%, about 18%, about 20%. about 25%, about 30%, or about 33% water.

The Ptx will crystallize on the balloon as the dihydrate.

### 4. Dehydrated PTx

The coated balloons as described in embodiment 3 may be heated at 50-100° C. for 24 hr. This results in dehydration of the PTx dihydrate.

### 5. PTx I/am

A medical device coated with PTx dihydrate or dehydrated (as described above) is heated to 175-195° C. resulting in the semicrystalline PTx Pam.

### 6. Amorphous Smooth Ptx Coating

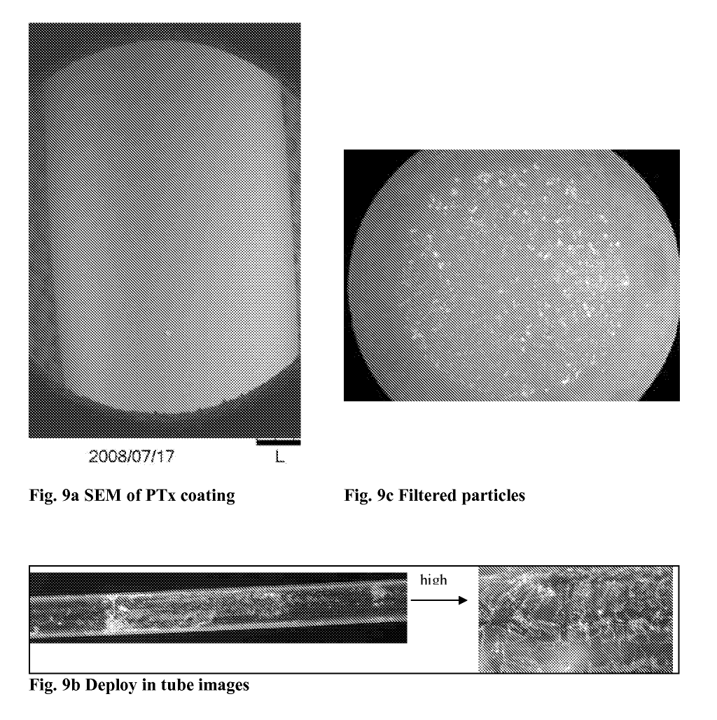

An inflated balloon (2.75×16 mm Liberte) is 1^{st} dip coated in a 10% solution of pectin in water and dried. The pectin acts as a dissolvable release layer. A 10% solids solution of Ptx in THF is prepared. The pectin coated balloon is dip coated into the Ptx solution. The Ptx coating is air dried then vacuum dried at room temperature. Ptx coat wt is 100-200 μg. The resulting coating is optically clear. The balloon is folded and deployed in a hydrophilic polyurethane tube using the following procedure. The tube is placed in water at 37° C. The folded balloon is placed in the tube and inflated after soaking for 1 min. The tube is sized to give overstretch during balloon deployment. Inflation is maintained for 1 minute, vacuum is pulled for 15 sec and the balloon is removed from the tube. The tube is removed from the water and dried and imaged. In another test a coated balloon is soaked in water at 37° C. for 1 min then deployed to 16 atm and immediately deflated. The water is immediately filtered to collect the particles given off the balloon during deployment. FIGS. 9a-9c, respectively, show SEM of the coated balloon, the tube after deployment and the filter after soak and deploy.

From FIGS. 9a-9c it can be seen that the Ptx coating is amorphous, continuous and micro smooth. Deployment in a tube results in large broken glass like, plate like particles.

### 7. Amorphous Porous Ptx Coating

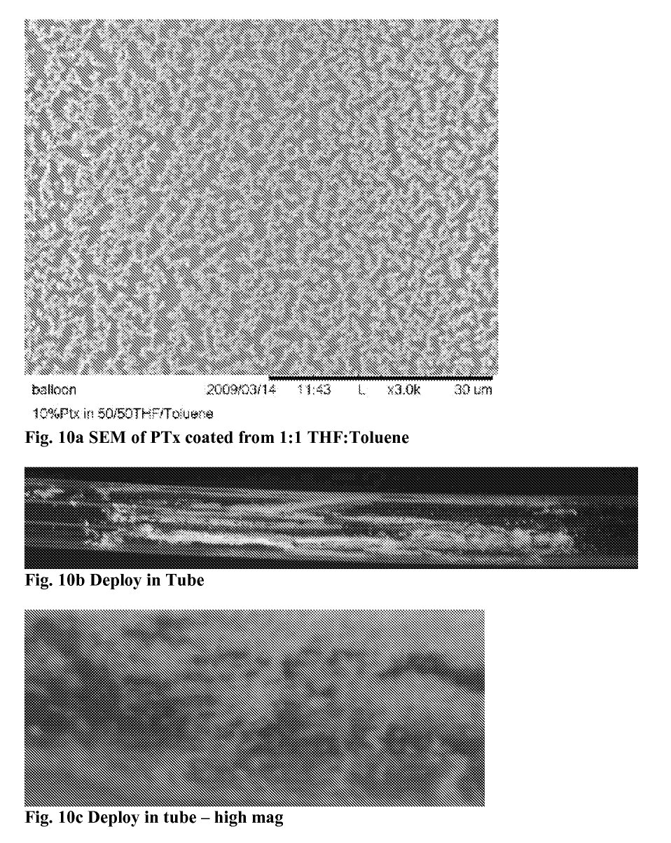

A balloon is dip coated in 10% PVP in IPA as a dissolvable base layer and dried. A 10% solution of Ptx in 1:1 THF:Toluene is prepared. The Ptx is completely soluble in the coating solution. THF is a fast evaporating, very good solvent for Ptx and Toluene is a slow evaporating poor solvent for Ptx. The balloon is dip coated in the PTx solution. The resulting dry coating is opaque white. The balloon is folded and tested as described in embodiment 6. Results are shown in FIGS. 10a-c.

SEM of the Ptx coating shows a microporous discontinuous coating. Rapid evaporation of the THF, post dip coating, results in phase separation of Ptx from the toluene solvent during the drying process leading to a discontinuous fine particle like coating. Deploy in tube and filtration after soak and deploy show fine particles deposited on the tube—in contrast to the large glassy, plate-like particles observed in embodiment 6.

8. Amorphous, Porous Ptx Coating from THF/Ethanol

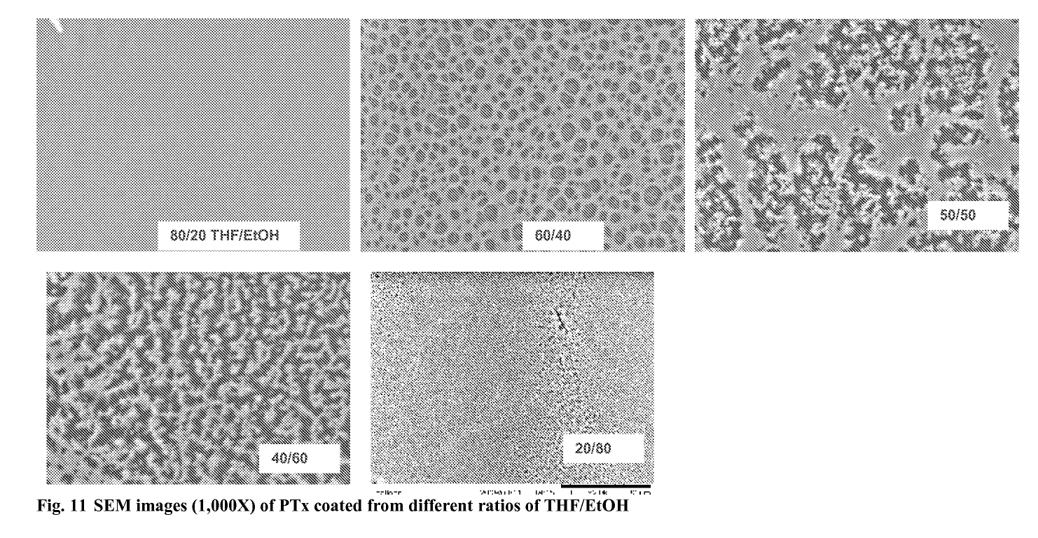

Solutions of 10% Ptx in THF/Ethanol (95%) were prepared. THF/EtOH ratio=80/20, 60/40, 50/50, 40/60. Ethanol is a slower evaporating poor solvent for PTx. Inflated Liberte balloons were dip coated in the solution. SEM was performed on the coated balloons. SEM images are shown in FIG. 11.

All solvent blends give different coating morphologies—from continuous (80/20), to semi-continuous (60/40), to microporous (40/60 and 20/80). All coatings appear amorphous.

9. Conversion of Ptx Coating from Amorphous to Crystalline

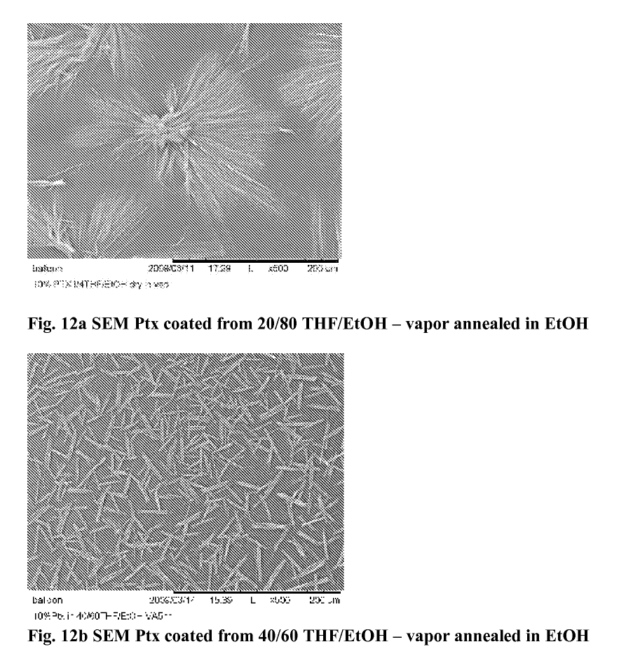

Ptx coated samples from embodiment 8 (20/80 and 40/60 THF/EtOH) were annealed in EtOH vapor in a sealed jar at RT for 4 hrs. FIGS. 12a and 12b show SEM images of the coatings after annealing.

The sample from 20/80 THF/EtOH shows well formed fan like Ptx crystals covering the balloon. The sample from 40/60 THF/EtOH shows discrete rod like crystals. The annealing process is effective at converting the DEB coating from amorphous Ptx to crystalline.

### 10. Amorphous Continuous Coating of PTx+Polyvinyl Pyrrolidinone (PVP) Excipient

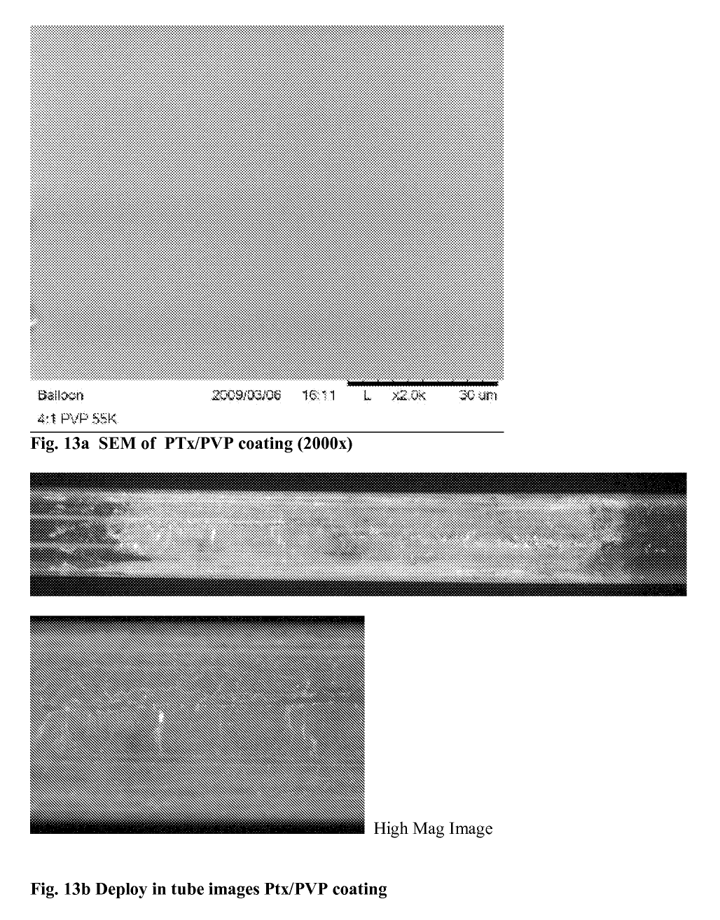

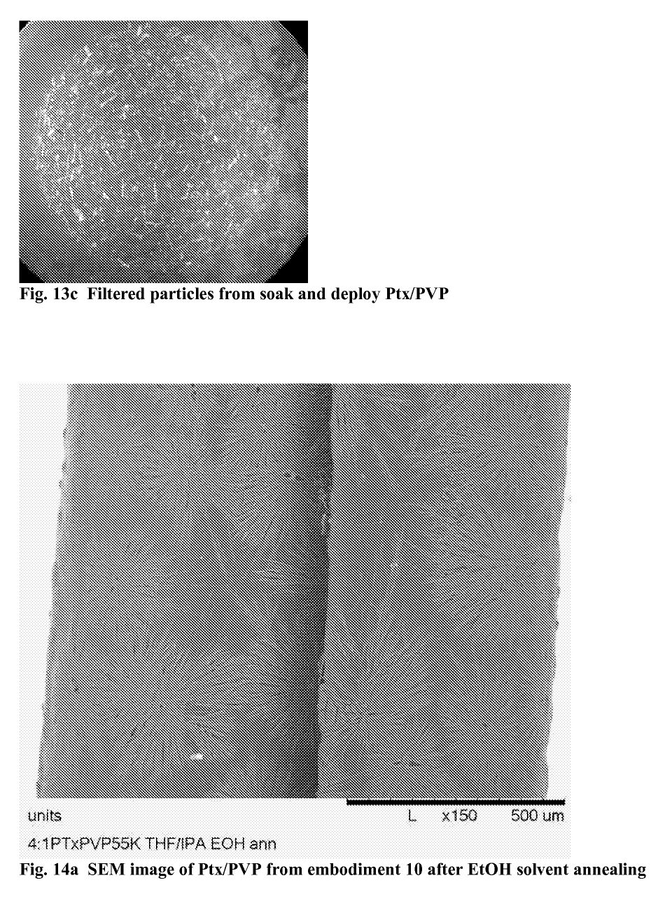

A 10% solution of 4:1 Ptx:PVP (wt:wt) in 4:1 THF:IPA (good solvent:fair solvent) was prepared. Balloons were dip coated and dried. The resulting coating was optically clear. The balloon was folded and tested as described in example 1. Results are shown in FIG. 13a-c.

SEM of the coated balloon shows a micro-smooth coating. Deploy in tube shows large regions of glassy film-like transfer to the tube. Filtered particles show large elongated particles.

### 11. Conversion of Amorphous Ptx to Crystalline PTx in PTx+PVP Coating

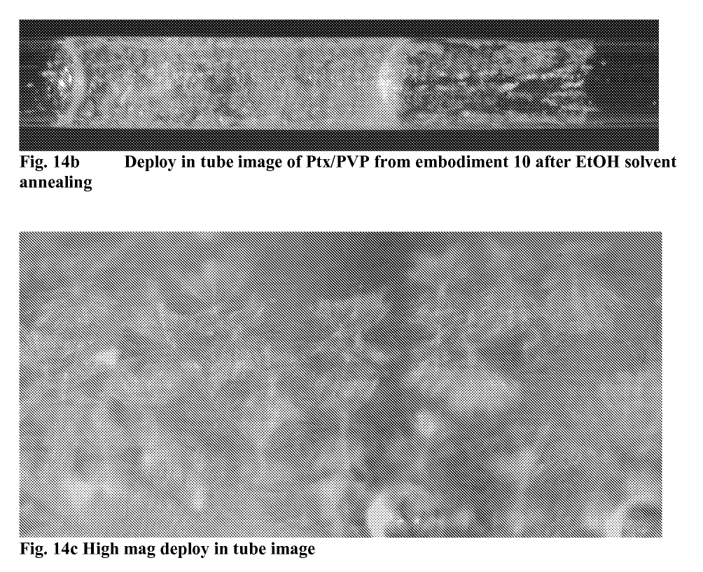

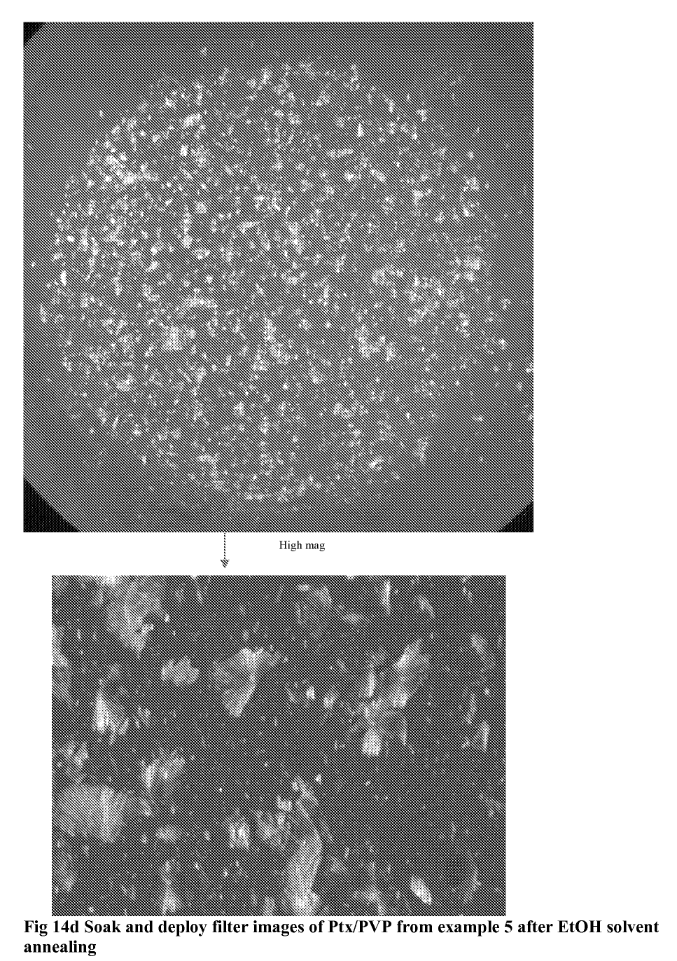

The amorphous sample from embodiment 10 was vapor annealed in EtOH for 4 hours. The balloon was folded and tested as described in embodiment 6. Results are shown in FIGS. 14a-d.

Vapor annealing converts the amorphous Ptx to fan like crystalline PTx in the PTx/PVP coating. Deploy in tube show transfer of crystalline PTx particles to the tube. Crystalline Ptx particles are also observed in the filtered soak and deploy sample.

### 12. Amorphous Micro-Porous Coating of PTx+PVP Excipient

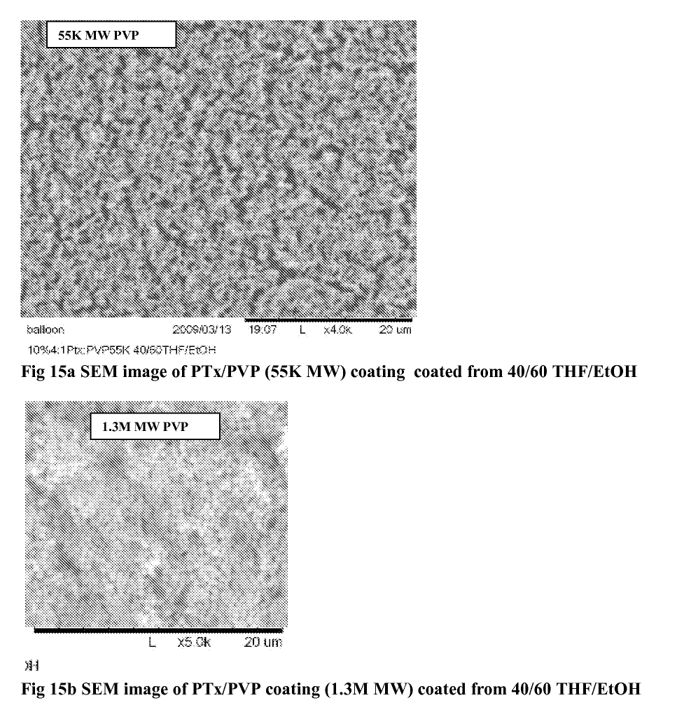

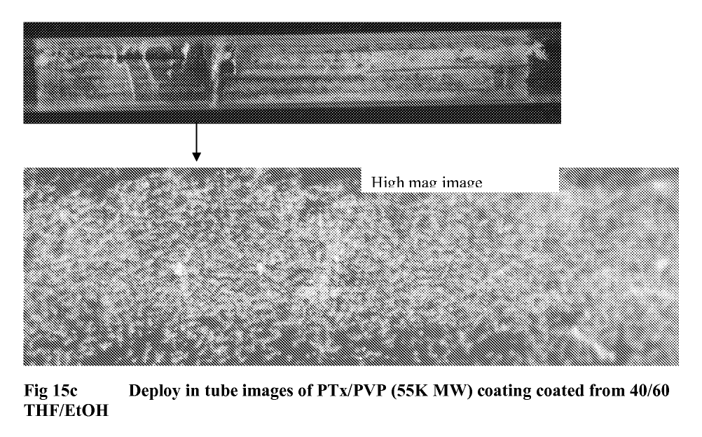

A 10% solution of 4:1 Ptx:PVP (wt:wt) in 40/60 THF: EtOH (good solvent:poor solvent) was prepared. Two different MW PVP's were used: Mw=55K and 1.3 million. Inflated balloons were dipped in the solution and air dried and then vacuumed dried. Coating wt was about 250 μg. The balloon was folded and tested as described in embodiment 6. Results are shown in FIG. 15.

SEM of the coated balloon show a micro-porous structure. The coating made with 1.3M MW PVP shows the coating is made up of ˜0.5 um diameter Ptx spherical particles. Deploy in tube shows transfer of fine Ptx particles, in contrast to large plate like particles for the same formulation (same ratio of PTx/PVP) coated from THF/IPA.

All published documents, including all US patent documents, mentioned anywhere in this application are hereby expressly incorporated herein by reference in their entirety. Any copending patent applications, mentioned anywhere in this application are also hereby expressly incorporated herein by reference in their entirety.

The above examples and disclosure are intended to be illustrative and not exhaustive. These examples and description will suggest many variations and alternatives to one of ordinary skill in this art. All these alternatives and variations are intended to be included within the scope of the claims, where the term “comprising” means “including, but not limited to”. Those familiar with the art may recognize other equivalents to the specific embodiments described herein which equivalents are also intended to be encompassed by the claims. Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all claims which possess all antecedents referenced in such dependent claim if such multiple dependent format is an accepted format within the jurisdiction. In jurisdictions where multiple dependent claim formats are restricted, the following dependent claims should each be also taken as alternatively written in each singly dependent claim format which creates a dependency from an antecedent-possessing claim other than the specific claim listed in such dependent claim.

1. (canceled)

2. A method of controlling tissue residence of a drug delivered by a transient device that is inserted into a body passageway, advanced through the body passageway to a treatment site and delivers drug to tissue at the site and is removed, wherein the drug has at least two morphological forms having different tissue residence characteristics, wherein the ratio of said morphological forms is controlled to provide therapeutically effective dosage at the site of delivery for a predetermined time after delivery.

3. A method as in claim 2 wherein the medical device is a balloon.

4. A method as in claim 2 wherein the drug is selected from the group consisting of paclitaxel, rapamycin, everolimus and mixtures thereof.

5. A method as in claim 2 wherein the drug is paclitaxel.

6. A method as in claim 2 wherein the drug has at least one amorphous morphological form and at least one crystalline amorphous form, and the drug is initially applied substantially in said amorphous form and at least a portion thereof is subsequently converted to said crystalline form.

7. A method as in claim 6 wherein said conversion comprises annealing the coating with a solvent vapor.

8. A method as in claim 2 wherein the drug is applied to the device as a formulation with an excipient.

9. (canceled)

10. A method as in claim 8 wherein said excipient is polyvinylpyrrolidone.

11. A method as in claim 2 wherein the ratio of said morphological forms of the drug is predetermined to provide a tissue residence of a therapeutically effective dosage for at least 5 days.

12. A method as in claim 11 wherein said ratio of said morphological forms of the drug is predetermined to provide a tissue residence of a therapeutically effective dosage for at least 10 days.

13. A method as in claim 2 wherein the drug comprises paclitaxel and said ratio is controlled to provide an amount of amorphous paclitaxel on the balloon of from 0-80 μg, an amount of anhydrous crystalline paclitaxel on the balloon of from 0-200 μg, and an amount of crystalline dihydrate paclitaxel on the balloon of from 50 to 1000 μg.

14-18. (canceled)

19. A drug coated balloon wherein the drug is paclitaxel or a mixture of paclitaxel and at least one other drug, the balloon having a selected distribution of at least two different morphological forms of paclitaxel thereon.

20. (canceled)

21. A drug coated balloon comprising a layer comprising paclitaxel wherein said layer comprises a crystalline form of paclitaxel in a water soluble polymer.

22. A drug coated balloon as in claim 21 wherein said water soluble polymer is polyvinylpyrrolidone.

23. A drug coated balloon as in claim 21 wherein said crystalline form of paclitaxel comprises crystalline paclitaxel dihydrate.

24. (canceled)

25. A drug coated balloon as in claim 19 wherein said drug is paclitaxel and comprises crystalline dihydrate paclitaxel.

26. (canceled)

27. A drug coated balloon as in claim 19 wherein the balloon is configured to deliver a dosage of paclitaxel predetermined to provide a tissue residence of a therapeutically effective dosage at the site of delivery for at least 5 days.

28. (canceled)

29. (canceled)

30. A drug coated balloon as in claim 19 wherein a fractional amount of from 1-25% of said paclitaxel is amorphous paclitaxel.

31. A drug coated balloon claim 19 wherein a fractional amount of from 1-25% of said paclitaxel is anhydrous crystalline paclitaxel.

32. A drug coated balloon as in claim 19 wherein a fractional amount of from 1-99% of said paclitaxel is dihydrate crystalline paclitaxel.
